(19) United States
(12) Patent Application Publication
(10) Pub. No.: US 2024/0382751 A1
(43) Pub. Date: Nov. 21, 2024

(54) CLINICIAN TASK PRIORITIZATION

(71) Applicant: Cochlear Limited, Macquarie University, NSW (AU)

(72) Inventors: Antonia Workman, Milsons Point, NSW (AU); Luka Denies, Utrecht (NL); Jan Patrick Frieding, Grose Vale, NSW (AU)

(21) Appl. No.: 18/692,015

(22) PCT Filed: Sep. 13, 2022
(86) PCT No.: PCT/IB2022/058619

§ 371 Date: Mar. 14, 2024

Related U.S. Application Data
(60) Provisional application No. 63/247,015, filed on Sep. 22, 2021.

(51) Int. Cl. A61N 1/36; A61N 1/05; A61N 1/372; G16H 40/67; G16H 50/70

(52) U.S. Cl. CPC A61N 1/36039; A61N 1/0541; A61N 1/36132; A61N 1/37247; G16H 40/67; G16H 50/70

(57) ABSTRACT

Presented herein are methods, systems and non-transitory computer readable storage media that provide for the configuration of medical devices. According to certain aspect of the present disclosure, a plurality of clinical data sets are obtained. Each of the plurality of clinical data sets is associated with a respective one of a plurality of recipients of a medical device. A relative priority of clinical support of the plurality of recipients is determined based, at least in part, on the plurality of clinical data sets. A prioritized clinician task list is displayed based on the relative priority of clinical support.

### BACKGROUND

### Field of the Invention

The present invention relates generally to techniques for prioritizing clinician tasks.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a first method is provided. The first method comprises obtaining a plurality of clinical data sets, wherein each of the plurality of clinical data sets is associated with a respective one of a plurality of recipients of a medical device; determining a relative priority of clinical support of the plurality of recipients based, at least in part, on the plurality of clinical data sets; and displaying, based on the relative priority of clinical support, a prioritized clinician task list.

In another aspect, a second method is provided. The second method comprises: obtaining clinical data associated with a plurality of clinical profiles, wherein each of the plurality of clinical profiles is associated with a different medical device recipient; analyzing the clinical data to determine a relative priority between the plurality of clinical profiles; and displaying, at a display screen, a prioritized task list in which the plurality of clinical profiles are organized based on the determined relative priority.

According to another aspect, a third method is provided. The third method comprises: providing a first clinician task list of clinical support for respective recipients of a medical device, wherein each item on the first clinician task list comprises a first relative priority determined based upon clinician-specific data and a first prioritizing model; obtaining an indication of a resolution provided by a clinician for an item on the first clinician task list; updating the first prioritizing model in response to obtaining the indication of the resolution provided by the clinician to generate a second prioritizing model; and providing a second clinician task list, wherein each item on the second clinician task list comprises a second relative priority determined based upon the clinician-specific data and the second prioritizing model.

According to other aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a comparison of clinician task prioritization for implanted medical devices generated according to the techniques of the present disclosure compared with related art techniques, in accordance with embodiments presented herein;

FIG. 3 is a flowchart of a first example method, in accordance with embodiments presented herein;

FIG. 4 is a schematic diagram illustrating a cochlear implant fitting system with which aspects of the techniques presented herein can be implemented;

FIG. 5 is a flowchart of a second example method, in accordance with embodiments presented herein;

FIG. 6 is a flowchart of a third example method, in accordance with embodiments presented herein;

FIG. 7 is a flowchart of a fourth example method, in accordance with embodiments presented herein;

FIG. 8 is a flowchart of a fifth example method, in accordance with embodiments presented herein;

FIG. 9 is a schematic diagram illustrating an implantable stimulation system with which aspects of the techniques presented herein can be implemented;

FIG. 10 is a schematic diagram illustrating a vestibular stimulator system with which aspects of the techniques presented herein can be implemented; and

FIG. 11 is a schematic diagram illustrating a retinal prosthesis system with which aspects of the techniques presented herein can be implemented.

### DETAILED DESCRIPTION

Presented herein are techniques for “configuring” or “fitting” medical devices, such as implantable medical devices, for a specific recipient. As explained in detail below, implantable medical devices are usually configured by medical professionals, referred to herein as clinicians, who are increasingly practicing remotely from the recipient/recipient in whom the implantable medical device is implanted. In operation, the clinicians receive data from recipient/implantable medical devices and, in turn, can determine operating parameters for the implantable medical device and return the parameters to the implantable medical device to configure, update, improve, or otherwise alter the operation of the implantable medical device. The techniques of the present disclosure provide for the generation and prioritizing of clinician tasks based, in part, on the operating parameters provided to the clinicians. Also, according to the techniques of the present disclosure, the generated tasks and their associated prioritizations may be modified based on clinician-specific data, such as clinician preferences, clinician technical expertise and/or clinician recipient lists. The techniques of the present disclosure also provide for the configuration of a model used to generate and modify clinician tasks using feedback from resolutions provided in response to previously generated tasks.

Merely for ease of description, the techniques presented herein are primarily described with reference to a specific implantable medical device system, namely a cochlear implant system. However, it is to be appreciated that the techniques presented herein may also be partially or fully implemented by other types of medical devices, both implantable and non-implantable. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

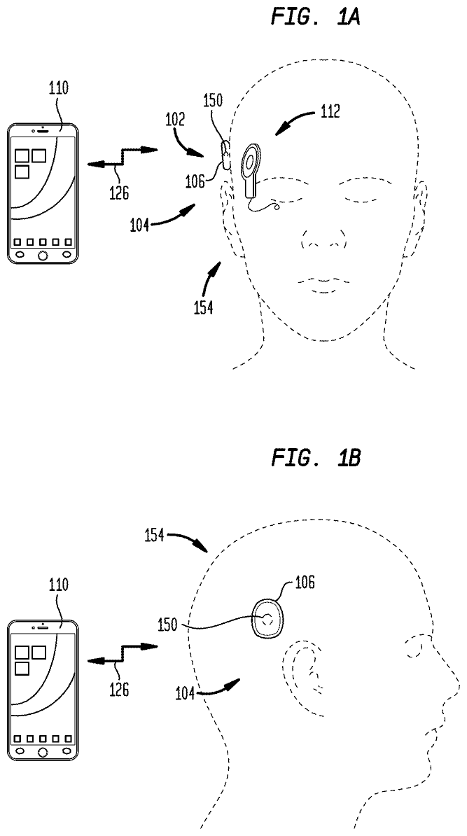

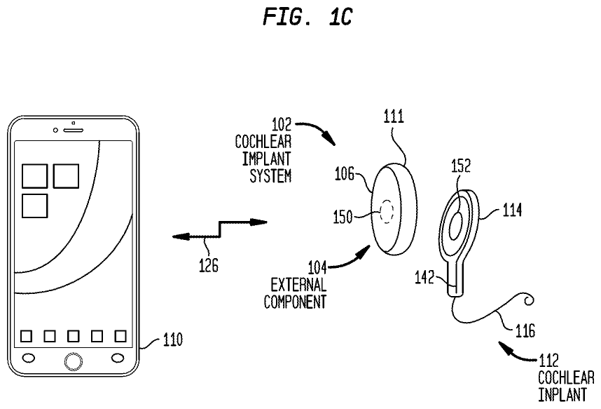

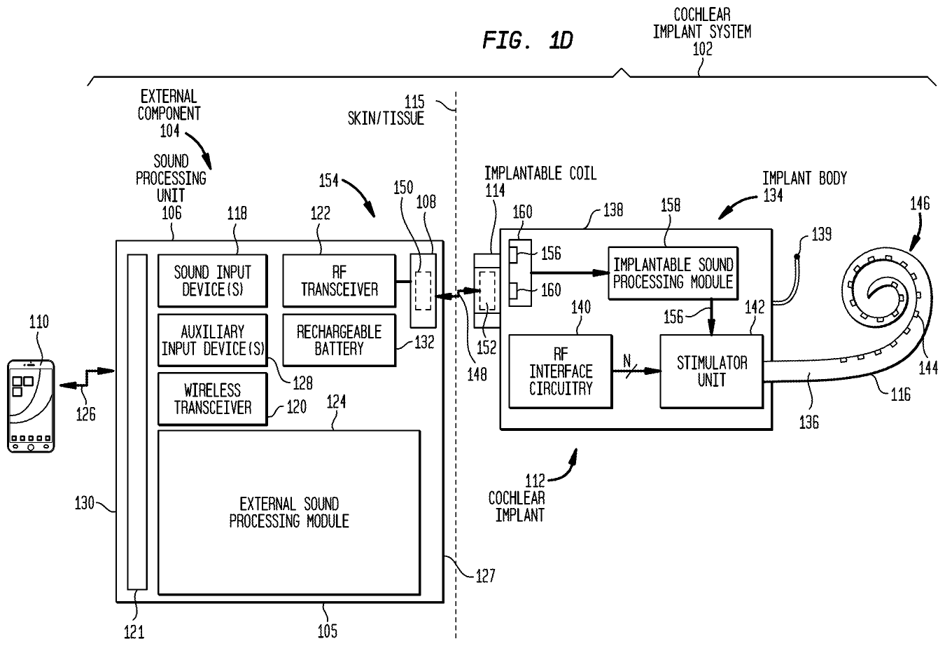

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted, cochlear implant system 102 includes one or more sound input devices 118 that receive electrical signals and/or convert audio signals into electrical input signals. The sound processing unit 106 processes the electrical input signals and generates stimulation data for use in delivering stimulation to the recipient in accordance with various operating parameters dictated by one of a number of selectable settings or modes of operation. The various selectable settings or modes of operation may be in the form of executable programs or sets of parameters for use in a program. The settings may accommodate any of a number of specific configurations that influence the operation of the cochlear implant. For example, the settings may include different digital signal and sound processing algorithms, processes and/or operational parameters for different algorithms, other types of executable programs (such as system configuration, user interface, etc.), or operational parameters for such programs. In certain examples, the selectable settings would be stored in a memory of the cochlear implant system 102 and relate to different optimal settings for different listening situations or environments encountered by the recipient (i.e., noisy or quite environments, windy environments, etc.).

Additionally, since the dynamic range for electrical stimulation is relatively narrow and varies across recipients and stimulating contacts, programs used in a sound processor are typically individually tailored to optimize the perceptions presented to a particular recipient (i.e., tailor the characteristics of electrical stimulation for each recipient). For example, many speech processing strategies rely on a customized set of stimulation settings which provide, for a particular recipient, the threshold levels (T-levels) and comfortable levels (C-levels) of stimulation for each frequency band. Once these stimulation settings are established, the sound processor may then optimally process and convert the received acoustic signals into stimulation data for use by the stimulator unit 142 in delivering stimulation signals to the recipient.

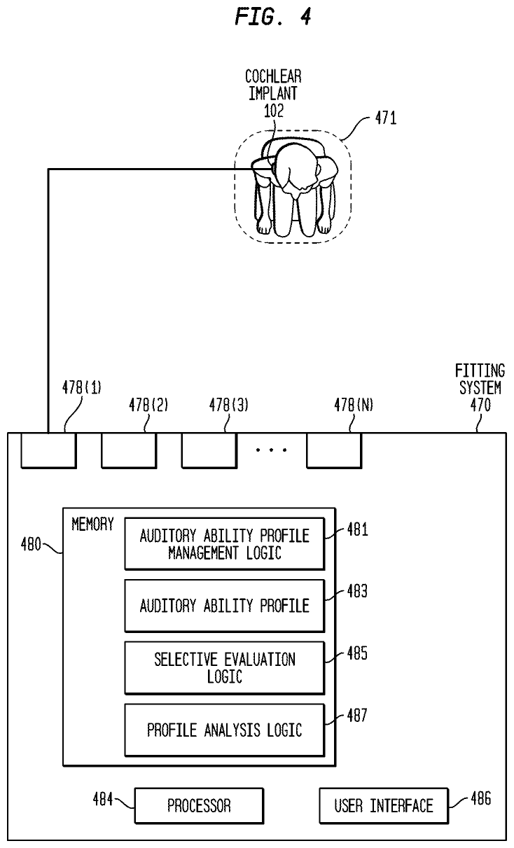

As such, it is clear that a typical cochlear implant has many parameters which determine the sound processing operations of the device. The individualized programs, commands, data, settings, parameters, instructions, modes, and/or other information that define the specific characteristics used by cochlear implant system 102 to process electrical input signals and generate stimulation data therefrom are generally and collectively referred to as “sound processing settings.” These parameters are determined and set by medical practitioners (clinicians) in a process known as “fitting” of the cochlear implant. As described in detail below with reference to FIG. 4, external device 110 may be configured to send data to a fitting system (see fitting system 470 of FIG. 4) via, for example, a computer network, such as a wide area network (WAN) or a local area network (LAN). This permits clinicians to use online technologies to individualize the programs, commands, data, settings, parameters, instructions, modes, and/or other information that define the specific characteristics used by cochlear implant system 102.

Traditionally, fitting of cochlear implants and other implantable medical devices was perform “in-person,” that is while the recipient visits the clinician's office. However, with the increased used in online technologies for healthcare services, clinicians now have the ability to perform “remote” fitting, where the clinician and recipient are at different locations. However, even with remote fitting, clinicians are expected to maintain the same standard of professional care regardless of whether they are consulting with recipients—in person or remotely/online. Outside of recipient consultations, clinicians are also expected to prepare recipient notes, organize prescriptions or referrals, manage their time scheduling and calendar, researching and other related tasks

Healthcare can be extended to include at home tasks and exercises to be carried out by recipients. In the case of a recipient with, for example, an implantable medical device, the practitioner has a role to indirectly supervise various at-home tests and/or rehabilitation exercises. As noted above, clinicians have also been enabled to fit or individualize/configure the performance of implantable medical devices remotely. However, clinicians are now grappling with how to prioritize and manage their limited attention to a growing number of and different kinds of clinical presentations, each having differing levels of demand on their attention, as well as clinical importance and urgency. These growing demands may leave the clinician disorganized as they try to determine how best to allocate their limited time, while still feeling assured their decisions are efficient from both a clinical perspective and a business perspective.

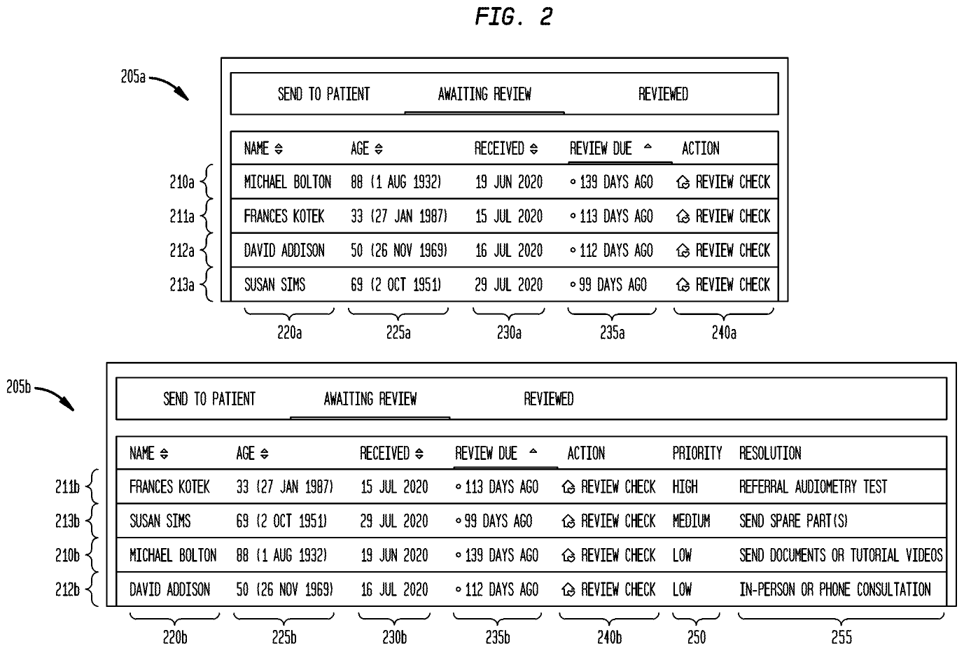

The techniques of the present disclosure present new ways to collect and interactively process various data types, and to create a new and practical system for supporting decision making in the new online environment. Accordingly, presented herein are methods, apparatuses and systems that provide tools for enabling clinicians to distribute or manage their limited resources, according to factors including clinical priorities of their recipient group, one example embodiment of which is contrasted with related art techniques in FIG. 2.

Specifically, illustrated in FIG. 2 are two user displays, user display 205a and user display 205b. User display 205a is generated through related art techniques, while user display 205b is generated according to an example embodiment of the techniques of the present disclosure. As shown, both user displays 205a and 205b include a listing of clinical tasks 210a-213a and 210b-213b, respectively. Each of these clinical tasks includes recipient name data 220a/220b, recipient age data 225a/225b, the date the clinical task was received 230a/230b, a proposed due date for review 235a/235b, and an action to be taken to resolve the task 240a/240b. User displays 205a and 205b differ in the sorting of clinical tasks 210a-213a and 210b-213b, respectively, and the inclusion of priority data 250 and suggested resolution data 255 in user display 205b. As shown in FIG. 2, where display 205a sorts clinical tasks 210a-213a by the order in which they were received, user display 205b sorts clinical tasks 210b-213b according to priority data 250.

As explained in detail below, the generation of user display 205b incorporates data from multiple sources in order to solve a specific problem faced by clinicians. More specifically, display 205b is generated by incorporating clinical data into determining the priority associated with clinical tasks 210b-213b, and ultimately how clinical tasks 210b-213b are sorted in display 205b. The generation of display 205b may also incorporate clinician specific data, such as clinician-specific preferences, expertise and recipient lists, in determining the priority data 250 for clinical tasks 210b-213b, and ultimately how clinical tasks 210b-213b should be sorted in display 205b. Additionally, the techniques of the present disclosure may incorporate feedback from the resolution of clinical tasks 210b-213b to train or update a model used to assign priority data 250 to clinical tasks. Accordingly, display 205b provides additional information that solves an identified problem for clinicians providing remote or telemedicine services for medical devices, including implantable medical devices.

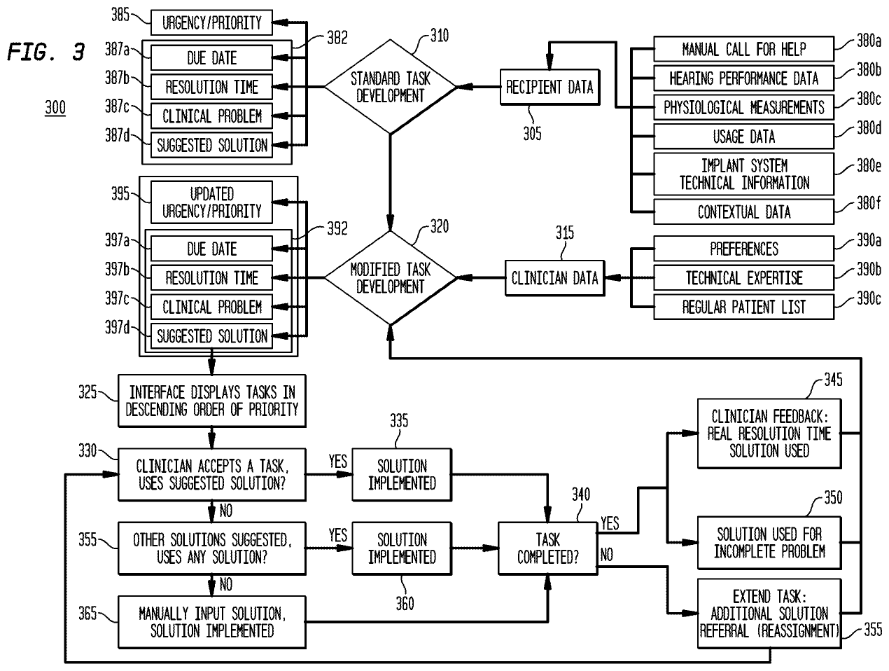

With reference now made to FIG. 3, depicted therein is a flowchart 300 illustrating a process flow according to the techniques of the present disclosure. More specifically, flowchart 300 illustrates three example embodiments of the techniques of the present disclosure, as well as the novel manner in which the three embodiments interact. Illustrated through operations 305, 310 and 325 is a first process via which a priority is assigned to a clinician task based upon receipt of clinical data 380a-f. Operations 315, 320 and 325 illustrate a second process via which a priority is assigned or updated based upon clinician-specific data 390a-c. Operations 330-360 illustrate a third process via which a clinician addresses prioritized clinician tasks, and provides feedback to the second process in order to configure and/or improve the updating and/or sorting of clinician tasks performed via operations 315, 320 and 325.

The first process begins in operation 305 where remote clinical data 380a-f is received at a computing device, such as a computing device utilized in a fitting system for an implantable medical device, a personal computer, a server computer (e.g., a server executing a database system and accompanying data processing functionality), a tablet or smart phone computing device, or other computing devices known to the skilled artisan. Turning briefly to FIG. 4, depicted therein is a block diagram illustrating an example fitting system 470 configured to execute the techniques presented herein. Fitting system 470 is, in general, a computing device that comprises a plurality of interfaces/ports 478(1)-478(N), a memory 480, a processor 484, and a user interface 486. The interfaces 478(1)-478(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 4, interface 478(1) is connected to cochlear implant system 102 having components implanted in a recipient 471. Interface 478(1) may be directly connected to the cochlear implant system 102 or connected to an external device that is communication with the cochlear implant systems. Interface 478(1) may be configured to communicate with cochlear implant system 102 via a wired or wireless connection (e.g., telemetry, Bluetooth, etc.).

The user interface 486 includes one or more output devices, such as a display screen (e.g., a liquid crystal display (LCD)) and a speaker, for presentation of visual or audible information to a clinician, audiologist, or other user. The user interface 486 may also comprise one or more input devices that include, for example, a keypad, keyboard, mouse, touchscreen, etc.

The memory 480 comprises auditory ability profile management logic 481 that may be executed to generate or update a recipient's auditory ability profile 483 that is stored in the memory 480. The auditory ability profile management logic 481 may be executed to obtain the results of objective evaluations of a recipient's cognitive auditory ability from an external device, such as an imaging system (not shown in FIG. 4), via one of the other interfaces 478(2)-478(N). In certain embodiments, memory 480 comprises subjective evaluation logic 485 that is configured to perform subjective evaluations of a recipient's cognitive auditory ability and provide the results for use by the auditory ability profile management logic 481. In other embodiments, the subjective evaluation logic 485 is omitted and the auditory ability profile management logic 481 is executed to obtain the results of subjective evaluations of a recipient's cognitive auditory ability from an external device (not shown in FIG. 4), via one of the other interfaces 478(2)-478(N).

The memory 480 further comprises profile analysis logic 487. The profile analysis logic 487 is executed to analyze the recipient's auditory profile (i.e., the correlated results of the objective and subjective evaluations) to identify correlated stimulation parameters that are optimized for the recipient's cognitive auditory ability.

Memory 480 may comprise read only memory (ROM), random access memory (RAM), magnetic disk storage media devices, optical storage media devices, flash memory devices, electrical, optical, or other physical/tangible memory storage devices. The processor 484 is, for example, a microprocessor or microcontroller that executes instructions for the auditory ability profile management logic 481, the subjective evaluation logic 485, and the profile analysis logic 487. Thus, in general, the memory 480 may comprise one or more tangible (non-transitory) computer readable storage media (e.g., a memory device) encoded with software comprising computer executable instructions and when the software is executed (by the processor 484) it is operable to perform the techniques described herein.

The correlated stimulation parameters identified through execution of the profile analysis logic 487 are sent to the cochlear implant system 102 for instantiation as the cochlear implant's current correlated stimulation parameters. However, in certain embodiments, the correlated stimulation parameters identified through execution of the profile analysis logic 487 are first displayed at the user interface 486 for further evaluation and/or adjustment by a user. As such, the user has the ability to refine the correlated stimulation parameters before the stimulation parameters are sent to the cochlear implant system 102.

The general operations for analysis of the recipient's auditory profile to identify correlated stimulation parameters that are optimized for the recipient's cognitive auditory ability have been described above. However, it is to be appreciated that the profile analysis logic 487 may operate in accordance with one or more selected guidelines set by a user via the user interface 486. For example, a user may configure the stimulation parameters that may be adjusted or set limits for how a stimulation parameter may be adjusted.

Returning to FIG. 3, the remote check data 380a-f received at the computing device of the fitting system in operation 305 may be received via the interfaces/ports 478(1)-(N) illustrated in FIG. 4. According to other example embodiments, data 380a-f may be received by another type of processing system or device, as described above. For example, data 380a-f may be received at a centralized database system that provides services according to the techniques of the present disclosure to multiple clinicians. According to other example embodiments, data 380a-f may be received at a clinician-specific database system configured to implement the techniques of the present disclosure. Such database systems may permit clinicians to access this data using separate computing devices, such as a fitting system 470 FIG. 4, a personal computing device, a tablet or smart phone computing device, or other computing devices known to the skilled artisan

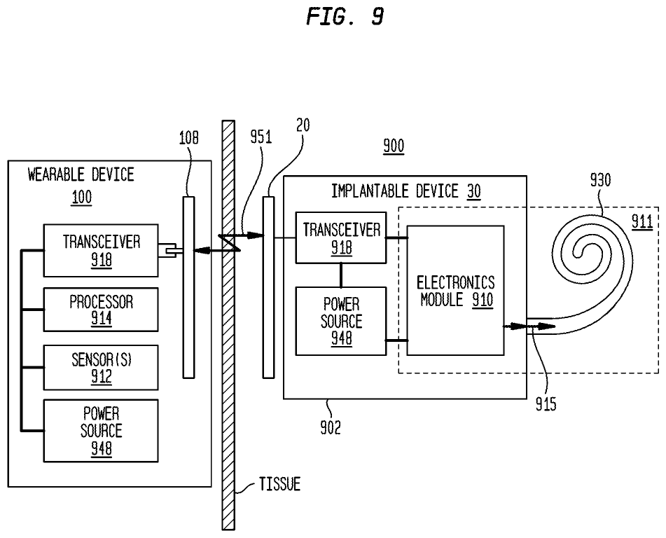

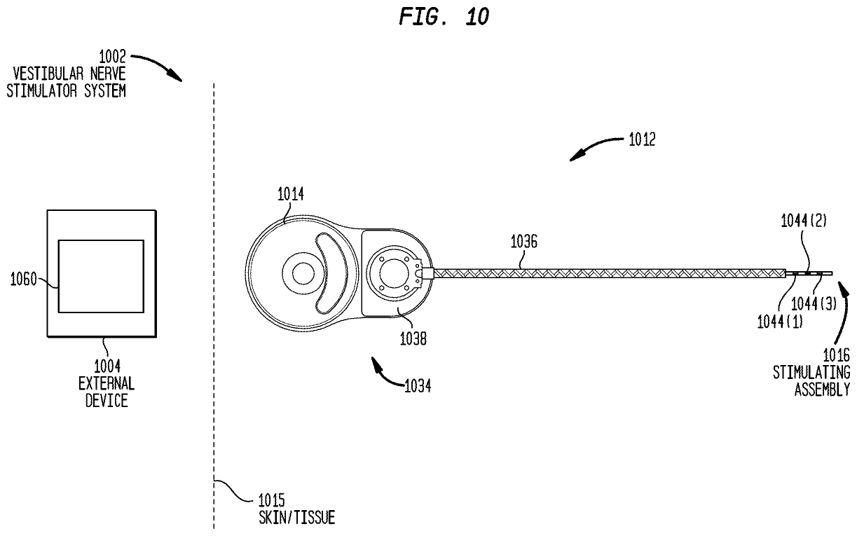

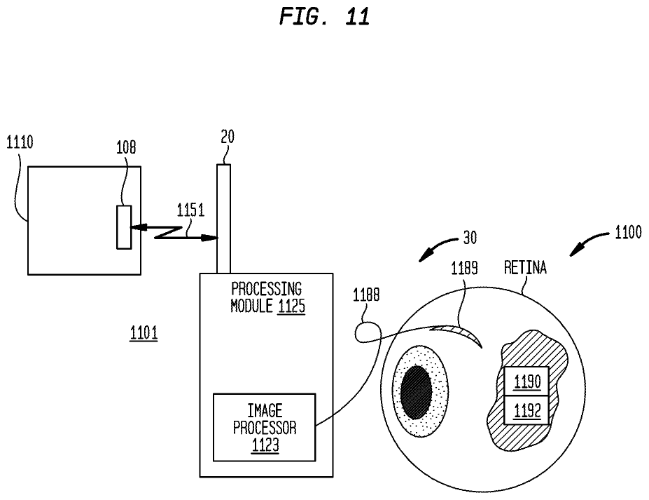

Additionally, the system that receives data 380a-f, and that implements the other operations of FIG. 3, need not be specific to one particular type of medical device. For example, a system according to the techniques of the present disclosure may receive data, such as data 380a-f, from a number of different medical device types, including implantable stimulation systems (see description accompanying FIG. 9, below), vestibular stimulator systems (see description accompanying FIG. 10, below), retinal prosthesis systems (see description accompanying FIG. 11, below), and other types of medical devices known to the skilled artisan. Accordingly, the processing device of FIG. 4 may be configured to communicate with multiple cochlear implant systems 102, as well as other types of medical devices.

Data 380a-f may be received via, for example, a fitting session during which a recipient's medical device interfaces with a fitting system, such as fitting system 470 of FIG. 4. According to other example embodiments, data 380a-f may be received in response to a recipient connecting their medical device (e.g., external component 104 of FIG. 1C) or associated external device (e.g., external device 110 of FIG. 1C) to the Internet. For example, a recipient's external device may be configured with an application, such as a smartphone “app,” that transfers data 380a-f to a processing system using the Internet. This transfer of data 380a-f may take place passively (e.g., without the recipient initiating the transfer) at regular intervals or in response to the external device connecting to the internet. According to other example embodiments, the transfer of data 380a-f may be actively initiated in response to a recipient or clinician command received at the external device.

The remote check data 380a-f includes manual call data 380a, hearing performance data 380b, physiological measurement data 380c, usage data 380d, implant system technical information data 380e and contextual data 380f. Manual call data 380a may include data sent via a user of a cochlear implant system, such as recipient 471 of cochlear implant system 102 of FIG. 4. Accordingly, manual call data 380a may be an embodiment of a request for assistance by a recipient of an implantable medical device. This manual call data 380a may include telephone audio data, text or instant message data, and/or email text data indicating one or more issues for a clinician to address in the cochlear implant system. Additionally, a recipient's external device (e.g., external device 110 of FIG. 1C) may be configured with an application (e.g., a smartphone “app”), that facilitates a recipient sending manual call data 380a.

Hearing performance data 380b may be embodied as a recipient's performance on audiometry tests. For example, if a recipient's performance on these tests has worsened more than 10% since the last check, the recipient may be struggling. Accordingly, such data may be used to prioritize tasks associated with such data with a higher priority. Physiological measurement data 380c may be embodied as data indicting if there are open/short circuits that have appeared since the last check. Such data may indicate that the recipient will need to come in for a re-programming appointment. Therefore, the techniques of the present disclosure may assign priority to these recipients or tasks with higher priority. Usage data 380d may be embodied as data indicating that a recipient is in the top percentile of coil-off events or the bottom percentile of on-air time. Accordingly, such recipients may not be getting much benefit from their implantable medical devices. Therefore, these cases may receive higher priority through the techniques of the present disclosure. Implant system technical information data 380e may be embodied as battery state data, error log data and/or implant reset data. Contextual data 380f may be embodied as data indicating when recipients received their medical implants. New implanted recipients are typically more worried about their performance. Therefore, newly implanted recipients may receive slightly higher priority than other recipients, based upon the techniques of the present disclosure. According to another embodiment, contextual data 380f may indicate checks that were longer ago, and therefore, these checks may cause tasks associated therewith to rise in priority, ensuring clinicians eventually address these tasks.

Hearing performance data 380b, physiological measurement data 380c, usage data 380d, implant system technical information data 380e and contextual data 380f may be considered fitting data, as this data may be used by a fitting system, such as fitting system 470 of FIG. 4. The data sets 380b-f may be automatically sent to the processing device (e.g., the processing devices described above, including a fitting system 470 of FIG. 4) via the cochlear implant system. According to other example embodiments, the fitting system may interface with the cochlear implant system to initiate the transfer of this data to the fitting system. According to still other example embodiments, the user of the cochlear implant system may initiate the sending of this data to the fitting system. FIG. 3 illustrates receipt of all of remote clinical data types 380a-f, but the techniques of the present disclosure are applicable to situations where only a subset of the illustrated clinical data types are received. For example, the techniques of the present disclosure are applicable to situations where only one type or some other subset of clinical data types 380a-f are received.

In operation 310, the remote clinical data 380a-f is used to generate a clinician task 382 with an associated urgency/priority 385. Operation 310 may apply remote clinical data 380a-f to a task generation model, such as a heuristic model or an artificial intelligence (AI) or machine learning based model such as a neural network. Based upon remote check data 380a-f, the model may generate clinician task 382 to include a due date 387a, a resolution time 387b, an identification of the clinical problem 387c, and a suggested solution 387d.

Task 382 and the accompanying urgency/priority 385 may be displayed on the display of the fitting system (e.g., user interface 486 of FIG. 4) in operation 325. While flowchart 300 illustrates intermediate operation 320 (to be described in detail below), the display of task 382 and accompanying urgency/priority 385 may be implemented directly from operation 310 in example embodiments in which operation 320 is not implemented.

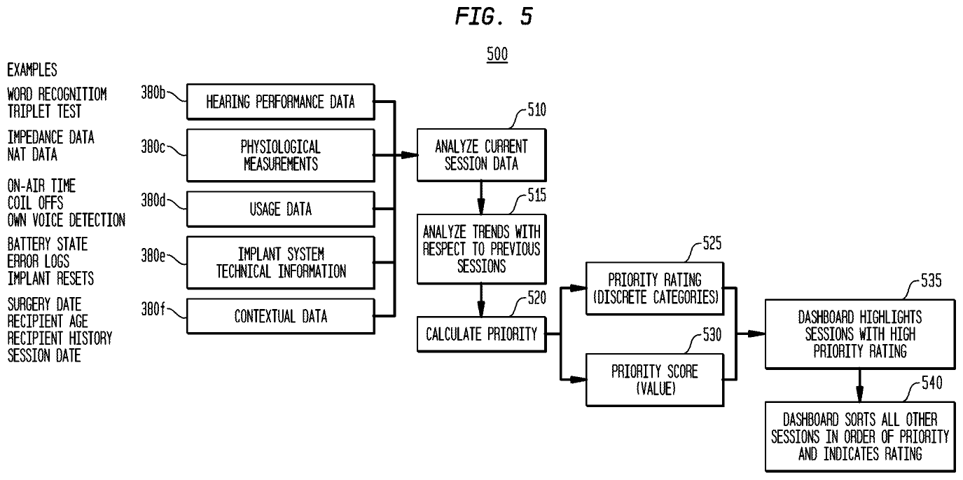

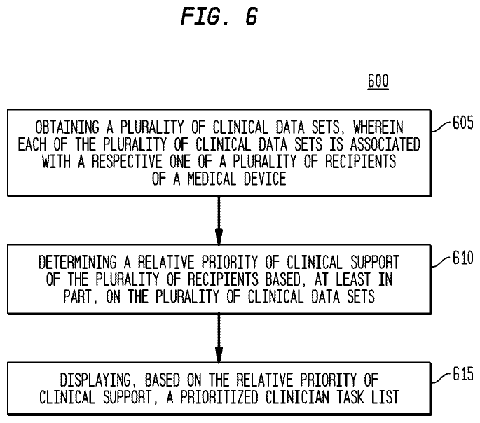

Turning to FIG. 5, depicted therein is a detailed example flowchart 500 for implementing the priority generation aspects of operations 310 and 320 of flowchart 300, as well as the priority display aspects of operation 325. Specifically, flowchart 500 begins with the receipt of remote clinical data 380b-f, which is analogous to remote clinical data 380b-f of FIG. 3. According to the specific example embodiment of FIG. 5, hearing performance data 380b is embodied as word recognition triplet test data, and physiological measurement data 380c is embodied as impedance rate Neural Response Telemetry (NRT) data. Usage data 380d is embodied as on-air time data, coil off data and/or recipient voice (i.e., “own voice”) detection data. Implant system technical data 380e is embodied as battery state data, error log data and/or implant reset data. Finally, contextual data 380f is embodied as data indicating the date of the implant surgery, the recipient's age, the recipient's history and a session date.

In operation 510, the clinical data 380b-f is analyzed, and in operation 515, trends are analyzed based on the data 380b-f as compared to previously received data analogous to that of data 380b-f.

Based on the analysis provided in operations 510 and 515, a priority for a clinical task is calculated or generated in operation 520. Alternatively, the priority may be calculated or generated for a clinical profile associated with a specific recipient. For example, a recipient of an implantable hearing prosthesis may have an associated fitting profile that indicates the individualized programs, commands, data, settings, parameters, instructions, modes, and/or other information that define the specific operating characteristics used by the recipient's hearing prosthesis. Accordingly, when the priority is calculated or generated, the priority may be associated with the recipient in general (or their clinical profile), as opposed to any one specific clinical task.

The priority generation provide by flowchart 500 may include determining a priority rating (e.g., “high,” “medium,” “low”) for the task in operation 525 and/or assigning the priority a value (e.g., a value from 1-100) in operation 530. Accordingly, operations 510-530 are analogous to the generation of the priority in operation 310 of FIG. 3.

Once the priority is generated in operations 510-530, operation 535 displays the task or clinical profile in a dashboard that highlights tasks or profiles with high priority, such as display 205b of FIG. 2. Finally, in operation 540, the dashboard sorts the tasks in order of priority (i.e., the priority value assigned in operation 530) and includes an indication of the task's rating (i.e., the rating assigned in operation 525), an example of which is illustrated in display 205b of FIG. 2. Accordingly operations 535 and 540 are analogous to operation 325 of FIG. 3.

As illustrated through operations 310 and 325 of FIG. 3 and/or flowchart 500 of FIG. 5, a display which lists clinical tasks based on priority, where the priority is derived from remote clinical data 380b-f, incorporates data from multiple sources in order to solve a specific problem faced by clinicians. More specifically, operations 310 and 325 of FIG. 3 and/or flowchart 500 of FIG. 5 generate their respective displays by incorporating clinical data into the display generation in determining the priority associated with clinical tasks, and ultimately how the clinical tasks should be sorted when displayed.

Returning to FIG. 3, in example embodiments where operation 320 is implemented, task 382 and the accompanying urgency/priority 385 may be modified in operation 320 to generate modified task 392 and accompanying modified urgency/priority 395 based upon clinician-specific data 390a-c. Specifically, in operation 315, clinician-specific data 390a-c is received at a computing device, such as the computing device utilized in the fitting system 470 of FIG. 4. According to the present example embodiment, operations 315 and 320 may be implemented at the same computing device as operations 305 and 310. Though, the skilled artisan will understand that operations 315 and 320 may be implemented at a computing device that is different from the computing device implementing operations 305 and 310.

The clinician data 390a-c received in operation 315 may include clinician preferences 390a, technical expertise data 390b and a regular recipient list 390c, though the skilled artisan will understand that the clinician-specific data received in operation 315 may include additional or alternative sources of clinician-specific data.

Clinician preferences 390a may include clinician-specific preferences for how clinician tasks should be resolved. Other clinician preferences 390a may include clinician-specific preferences for how clinician tasks should be prioritized. For example, clinician preferences 390a may indicate that tasks addressing a first clinical problem or associated with a first patent should be prioritized higher or lower relative to tasks of a second clinical problem or associated with a second recipient. As a further example, preferences 390a may indicate an absolute priority associated with a particular recipient or clinical problem should be assigned a highest or lowest priority, regardless of the priorities associated with other tasks assigned to the clinician.

Technical expertise data 390b may indicate areas of particular expertise for the associated clinician. For example, if a clinician has specialized skills in a particular technical area, clinician tasks requiring such particularized skills may be assigned a higher priority. Additionally, tasks particularly relevant to the clinician's technical expertise may be modified based on this expertise. For example, the suggested solution for a particular task may be changed to a solution that the specific clinician may provide based on their technical expertise, a solution that other clinicians with different technical expertise may not be able to provide.

Regular recipient list 390c is, as its name suggests, a list of regular recipients for a particular clinician. Tasks may be assigned higher or lower priority based upon whether or not the recipient associated with the task is listed on the regular recipient list. For example, some clinicians may intend to prioritize new recipients, while other clinicians may intend to prioritized established recipients. Accordingly, the presence or lack thereof of a recipient on the regular recipient list 390c may result in a modification to a task or priority/urgency.

Other clinician data received in operation 315 may include a clinician's current schedule of recipients or calendar, a list of a clinician's partners, a list of a clinician's resources, such as additional diagnostic equipment, and other types of data known to the skilled artisan.

Next, operation 320 applies a task modification model to the task 382 and/or urgency/priority 385 generated in operation 310. The task modification model of operation 320 is configured to evaluate task 382 and/or urgency priority 385, and modify them based, in part, on the clinician-specific data 390a-c received in operation 315. For example, the task modification model of operation 320 may modify one or more of the due date 387a, the resolution time 387b, the identification of the clinical problem 387c, or the suggested solution 387d of task 382, resulting in the generation of modified task 392. One or more of modified due date 397a, modified resolution time 397b, modified identification of the clinical problem 397c, or modified suggested solution 397d of modified task 392 may differ from those of the corresponding values in task 382. The task modification model may also modify the urgency/priority 385 to generate modified urgency/priority 395. This is not to say that the application of the task modification model must modify one or more values in either of task 382 or urgency/priority 385. The novel aspect of the task modification model of operation 320 is in the application of the model and the potential to update task 382 based upon, in part, the clinician data received in operation 315.

The task modification model of operation 320 may be embodied as a heuristic model or an AI or machine learning based model, such as a neural network. Furthermore, as explained in greater detail below, the task modification model of operation 320 may be updated or trained based on clinician feedback received via operations 345, 350 and/or 355.

Next, in operation 325, modified task 392 and the accompanying modified urgency/priority 395 are displayed via operation 325. More specifically, operation 325 displays tasks in a user interface display in descending order of urgency/priority. By providing modified tasks, such as modified task 392, in order of priority/urgency specific to the clinician, the display of operation 325 represents a specific display that incorporates data from multiple sources in order to solve a problem faced by clinicians. As noted above, clinicians are now grappling with how to prioritize and manage their limited attention to a growing number of and different kinds of clinical presentations, each having differing levels of demand on their attention, as well as clinical importance and urgency. This situation can leave the clinician disorganized as they try to determine how best to allocate their limited time, while still feeling assured their decisions are efficient from both a clinical perspective and a business perspective. By generating a display that includes an indication of urgency/priority specific to the clinician's preferences, expertise and recipients, the display of operation 325 overrides the routine and conventional sequence of display generation that is based solely on, for example, the order in which recipients have reported their problems.

Furthermore, because the display of operation 325 provides an indication of urgency/priority that is specific to the clinician and its preferences, expertise, and recipients, the display of operation 325 provides additional information that solves an identified problem for clinicians providing remote or telemedicine services for implantable medical devices. Related art techniques for displaying clinician tasks base urgency on the timing of the reporting of the problem, or at best, based on the type of problem reported. The display of operation 325, through its use of clinician specific urgency/priority, provides additional information, mainly how the specific clinician would prioritize the task based on their specific preferences, expertise, and recipients. Accordingly, the display of operation 325 represents a specific way of automating the creation of clinician task lists and solves the particular problem of a clinician having to manually prioritize and organize their work using a combination of standard office software and personal judgment. Furthermore, the display of operation 325 improves upon related art task display systems by providing a system that provides for filtering or sorting of clinician task content on an individually customizable basis. As such, the display of operation 325 is directed to the practical application of providing clinicians with a display that includes an indication of urgency/priority that is specific to the clinician and its preferences, expertise, and recipients.

Once provided with a display via operation 325, a clinician accepts a task in operation 330, and decides whether to use the suggested solution 397d. If the clinician accepts the suggested solution 397d, the solution is implemented in operation 335.

If, on the other hand, the clinician does not use selected solution 397d, the clinician may decide to implement another suggested solution in operation 355. If the clinician opts for the other suggested solution, the solution is implemented in operation 360.

If the clinician does not opt for any of the suggested solutions, the clinician manually inputs a solution and the solution is implemented in operation 365.

Regardless of which solution is implemented, the suggested solution of operation 330, the other suggested solution of operation 355, or the manually entered solution of operation 360, the implementation of the solution may include the sending of configuration data and/or operating parameters directly to an implantable medical device, such as the cochlear implant system 102 of FIG. 4. For example, the implementation of the solution in any one of operations 335, 360 or 365, may include the sending of correlated stimulation parameters to the cochlear implant system. Accordingly, the techniques illustrated in FIG. 3 include the configuration of the operating parameters of an implantable medical device, such as a cochlear implant system.

In operation 340, subsequent to the implementation of the solution, a determination is made regarding whether or not the task is completed. For example, the solution implemented in operations 335, 360 or 365 may be sufficient to resolve the clinical problem 397c associated with a modified task 392. If the task is completed, e.g., the clinical problem 397c is resolved, then operation 345 results in clinician feedback being provided to the task modification model utilized in operation 320. More specifically, the feedback provided in operation 345 may include the real resolution time and the solution used. If, on the other hand, the task is not completed, e.g., the clinical problem 397c remains unresolved, operation 350 provides feedback to the task modification model utilized in operation 320 indicating the solution used and that the task remains incomplete, and the clinical problem 397c remains unresolved. If the task is extended, meaning the task remains incomplete, operation 355 may include reassigning the task, essentially sending the task back to the display of operation 325 so that it may be re-accepted by the clinician in a subsequent instance of operation 330. Operation 355 may also include providing feedback to the task modification model utilized in operation 320 indicating that the task remains incomplete and requires an additional solution referral.

Through the feedback provided in operations 345, 350, and 355, the task modification model utilized in operation 320 may be improved. For example, if the task modification model utilized in operation 320 is embodied as an AI or machine learning based model, the feedback provided in operations 345, 350, and 355 allows for the retraining of the task modification model such that it takes into account the clinician decisions that resolved or did not resolve a particular task. For example, the feedback provided in operations 345, 350, and 355 may be used to generate or supplement training sets used to train the task modification model utilized in operation 320. By retraining the task modification model utilized in operation 320 using this feedback, the task modification model's operation may be improved, providing more accurate modified urgencies/priorities 395, modified due dates 397a, modified resolution times 397b, modified identifications of the clinical problem 397c, or modified suggested solutions 397d. Accordingly, the feedback of operations 345, 350, and 355 improves the functioning of the computing device implementing operation 320.

In summary, flowchart 300 provides a robust system that supports decision making in environments where online technologies for healthcare services are used that allow clinicians to maintain the same standard of professional care regardless of whether they are consulting with recipients in person or online.

With reference now made to FIG. 6, depicted therein is a flowchart 600 of an example method in accordance with the techniques provided herein. More specifically, flowchart 600 illustrates a method of displaying a prioritized task list organized based on relative priority. For example, the method of flowchart 600 may represent a method for generating the display of operation 325 of FIG. 3.

Flowchart 600 begins in operation 605 where a plurality of clinical data sets are obtained. Each of the clinical data sets obtained in operation 605 is associated with a respective one of a plurality of recipients of a medical device. For example, operation 605 may be embodied as the receipt of one or more data sets 380b-f in operation 305 of FIG. 3 or receipt of one or more data sets 380b-f in FIG. 5.

In operation 610, a relative priority of clinical support of the plurality of recipients is determined. The determination of the relative priority is based, at least in part, on the plurality of clinical data sets. For example, operation 610 may be embodied as one or both of operations 310 or 320 of FIG. 3, where urgency/priorities are generated or modified for clinician tasks.

Finally, in operation 615, a prioritized clinician task list is displayed. The displaying is based on the relative priority of clinical support. For example, operation 615 may be embodied as the display of operation 325 of FIG. 3 or the display and sorting provided by in operations 535 and 540 of FIG. 5.

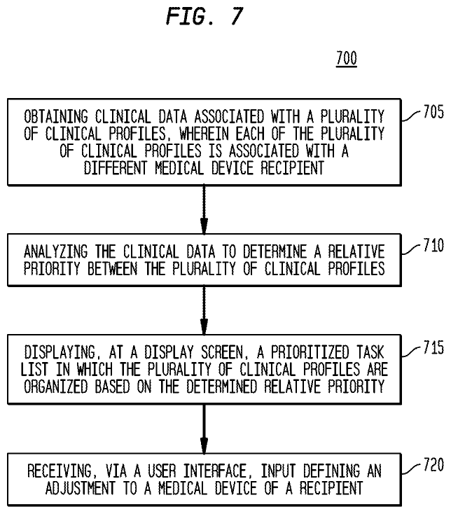

With reference now made to FIG. 7, depicted therein is a flowchart 700 of a second example method in accordance with the techniques provided herein. Flowchart 700 begins in operation 705 where clinical data is obtained. The clinical data is associated with a plurality of clinical profiles, and each of the plurality of clinical profiles is associated with a different medical device recipient. For example, recipients of implanted hearing prostheses may have an associated clinical profile that indicates the individualized programs, commands, data, settings, parameters, instructions, modes, and/or other information that define the specific operating characteristics used by the recipient's hearing prosthesis. Accordingly, the clinical data received in operation 705 may be associated with a clinical profile when, for example, the clinical data indicates a performance parameter associated with the medical device that operates according to the clinical profile. Operation 705 may be embodied as the receipt of one or more data sets 380b-f in operation 305 of FIG. 3 or receipt of one or more data sets 380b-f in FIG. 5.

In operation 710, the clinical data is analyzed to determine a relative priority between the plurality of clinical profiles. Example embodiments of operation 710 may be one or more of operations 310 or 320 of FIG. 3. Example embodiments of operation 710 may also be operations 510-520 of FIG. 5.

Next, in operation 715, a prioritized task list is displayed in which the plurality of clinical profiles are organized based on the determined relative priority. Example embodiments of operation 715 may include operation 325 of FIG. 3 or operations 535 and 540 of FIG. 5.

Also illustrated in FIG. 7 is an optional operation 720 in which input defining an adjustment to a medical device of a recipient is received via a user interface. Data indicative of the adjustment may also be communicated to the medical device and/or to the recipient. According to one specific example embodiment, the adjustment may be communicated to the medical device and/or the recipient via, for example, a computer network, as discussed above with reference to FIG. 4.

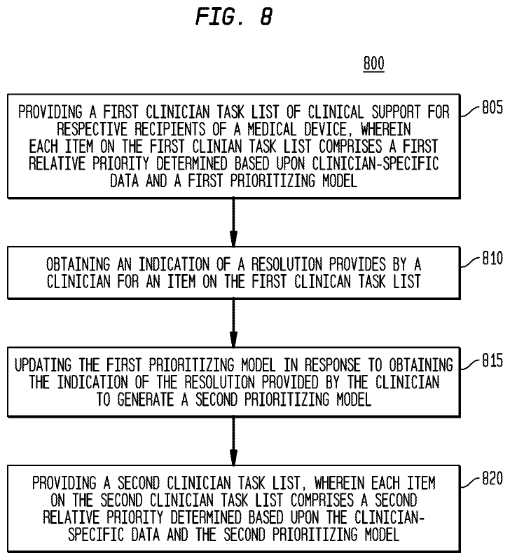

With reference now made to FIG. 8, depicted therein is a flowchart 800 of a third example method in accordance with the techniques provided herein. Flowchart 800 begins in operation 805 where a first clinician task list is provided. The first clinician task list includes items for clinical support for respective recipients of a medical device. Each item on the first clinician task list includes a first relative priority determined based upon clinician-specific data and a first prioritizing model. Accordingly, operation 805 may be embodied as the display provided in operation 325 of FIG. 3.

In operation 810, an indication of a resolution provided by the clinician for an item on the first clinician task list is obtained. Accordingly, operation 810 may be embodied as one or more of operations 345, 350 or 355 of FIG. 3.

In operation 815, the first prioritizing model is updated in response to obtaining the indication of the resolution provided by the clinician. The updating of the first prioritizing model results in the generation of a second prioritizing model. Accordingly, operation 815 may be embodied as the updating of the model used in operation 320 of FIG. 3 based upon the feedback provided in one or more of operations 345, 350 or 355.

Finally, in operation 820, a second clinician task list is provided. Each item on the second clinician task list comprises a second relative priority determined based upon the clinician-specific data and the second prioritizing model. Accordingly, operation 820 may be embodied as operation 325 of FIG. 3 in which a task list is updated after the model used in operation 320 is updated in response to the feedback provided in one or more of operations 345, 350 of 355.

In summary, by implementing one or more of the methods provided above with reference to FIGS. 4-8, clinicians may be enabled to focus on recipients who need their help most, and clinicians may be better able to plan their work, ensuring that tasks with low priority will typically take less time than those with high priority that require follow-up. Recipients who need it receive care more quickly, because those with higher priority problems are flagged to the clinician. Furthermore, clinicians will be better equipped to deal with an increasing number of recipients as they can prioritize remote checks with increased efficiency

As previously described, the technology disclosed herein can be applied in any of a variety of circumstances and with a variety of different devices. Example devices that can benefit from technology disclosed herein are described in more detail in FIGS. 9-11, below. As described below, the operating parameters for the devices described with reference to FIGS. 9-11 may be configured using a fitting system analogous to fitting system 470 of FIG. 4. For example, the techniques described herein can be used to prioritize clinician tasks associated with configuring the operating parameters of wearable medical devices, such as an implantable stimulation system as described in FIG. 9, a vestibular stimulator as described in FIG. 10, or a retinal prosthesis as described in FIG. 11. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 9 is a functional block diagram of an implantable stimulator system 900 that can benefit from the technologies described herein. The implantable stimulator system 900 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 902. Here, the wearable device 100 is configured to transcutaneously couple with the implantable device 30 via a wireless connection to provide additional functionality to the implantable device 30.

In the illustrated example, the wearable device 100 includes one or more sensors 912, a processor 914, a transceiver 918, and a power source 948. The one or more sensors 912 can be one or more units configured to produce data based on sensed activities. In an example where the stimulation system 900 is an auditory prosthesis system, the one or more sensors 912 include sound input sensors, such as a microphone, an electrical input for an FM hearing system, other components for receiving sound input, or combinations thereof. Where the stimulation system 900 is a visual prosthesis system, the one or more sensors 912 can include one or more cameras or other visual sensors. Where the stimulation system 900 is a cardiac stimulator, the one or more sensors 912 can include cardiac monitors. The processor 914 can be a component (e.g., a central processing unit) configured to control stimulation provided by the implantable device 30. The stimulation can be controlled based on data from the sensor 912, a stimulation schedule, or other data. Where the stimulation system 900 is an auditory prosthesis, the processor 914 can be configured to convert sound signals received from the sensor(s) 912 (e.g., acting as a sound input unit) into signals 951. The transceiver 918 is configured to send the signals 951 in the form of power signals, data signals, combinations thereof (e.g., by interleaving the signals), or other signals. The transceiver 918 can also be configured to receive power or data. Stimulation signals can be generated by the processor 914 and transmitted, using the transceiver 918, to the implantable device 30 for use in providing stimulation.

In the illustrated example, the implantable device 30 includes a transceiver 918, a power source 948, and a medical instrument 911 that includes an electronics module 910 and a stimulator assembly 930. The implantable device 30 further includes a hermetically sealed, biocompatible implantable housing 902 enclosing one or more of the components.

The electronics module 910 can include one or more other components to provide medical device functionality. In many examples, the electronics module 910 includes one or more components for receiving a signal and converting the signal into the stimulation signal 915. The electronics module 910 can further include a stimulator unit. The electronics module 910 can generate or control delivery of the stimulation signals 915 to the stimulator assembly 930. In examples, the electronics module 910 includes one or more processors (e.g., central processing units or microcontrollers) coupled to memory components (e.g., flash memory) storing instructions that when executed cause performance of an operation. In examples, the electronics module 910 generates and monitors parameters associated with generating and delivering the stimulus (e.g., output voltage, output current, or line impedance). In examples, the electronics module 910 generates a telemetry signal (e.g., a data signal) that includes telemetry data. The electronics module 910 can send the telemetry signal to the wearable device 100 or store the telemetry signal in memory for later use or retrieval.

The stimulator assembly 930 can be a component configured to provide stimulation to target tissue. In the illustrated example, the stimulator assembly 930 is an electrode assembly that includes an array of electrode contacts disposed on a lead. The lead can be disposed proximate tissue to be stimulated. Where the system 900 is a cochlear implant system, the stimulator assembly 930 can be inserted into the recipient's cochlea. The stimulator assembly 930 can be configured to deliver stimulation signals 915 (e.g., electrical stimulation signals) generated by the electronics module 910 to the cochlea to cause the recipient to experience a hearing percept. In other examples, the stimulator assembly 930 is a vibratory actuator disposed inside or outside of a housing of the implantable device 30 and configured to generate vibrations. The vibratory actuator receives the stimulation signals 915 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 918 can be components configured to transcutaneously receive and/or transmit a signal 951 (e.g., a power signal and/or a data signal). The transceiver 918 can be a collection of one or more components that form part of a transcutaneous energy or data transfer system to transfer the signal 951 between the wearable device 100 and the implantable device 30. Various types of signal transfer, such as electromagnetic, capacitive, and inductive transfer, can be used to usably receive or transmit the signal 951. The transceiver 918 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 948 can be one or more components configured to provide operational power to other components. The power source 948 can be or include one or more rechargeable batteries. Power for the batteries can be received from a source and stored in the battery. The power can then be distributed to the other components as needed for operation.

As should be appreciated, while particular components are described in conjunction with FIG. 9, technology disclosed herein can be applied in any of a variety of circumstances. The above discussion is not meant to suggest that the disclosed techniques are only suitable for implementation within systems akin to that illustrated in and described with respect to FIG. 9. In general, additional configurations can be used to practice the methods and systems herein and/or some aspects described can be excluded without departing from the methods and systems disclosed herein.

FIG. 10 illustrates an example vestibular stimulator system 1002, with which embodiments presented herein can be implemented. As shown, the vestibular stimulator system 1002 comprises an implantable component (vestibular stimulator) 1012 and an external device/component 1004 (e.g., external processing device, battery charger, remote control, etc.). The external device 1004 comprises a transceiver unit 1060. As such, the external device 1004 is configured to transfer data (and potentially power) to the vestibular stimulator 1012,

The vestibular stimulator 1012 comprises an implant body (main module) 1034, a lead region 1036, and a stimulating assembly 1016, all configured to be implanted under the skin/tissue (tissue) 1015 of the recipient. The implant body 1034 generally comprises a hermetically-sealed housing 1038 in which RF interface circuitry, one or more rechargeable batteries, one or more processors, and a stimulator unit are disposed. The implant body 134 also includes an internal/implantable coil 1014 that is generally external to the housing 1038, but which is connected to the transceiver via a hermetic feedthrough (not shown).

The stimulating assembly 1016 comprises a plurality of electrodes 1044(1)-(3) disposed in a carrier member (e.g., a flexible silicone body). In this specific example, the stimulating assembly 1016 comprises three (3) stimulation electrodes, referred to as stimulation electrodes 1044(1), 1044(2), and 1044(3). The stimulation electrodes 1044(1), 1044(2), and 1044(3) function as an electrical interface for delivery of electrical stimulation signals to the recipient's vestibular system.

The stimulating assembly 1016 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 1012, the external device 1004, and/or another external device, can be configured to implement the techniques presented herein. That is, the vestibular stimulator 1012, possibly in combination with the external device 1004 and/or another external device, can include an evoked biological response analysis system, as described elsewhere herein.

FIG. 11 illustrates a retinal prosthesis system 1101 that comprises an external device 1110 (which can correspond to the wearable device 100) configured to communicate with a retinal prosthesis 1100 via signals 1151. The retinal prosthesis 1100 comprises an implanted processing module 1125 (e.g., which can correspond to the implantable device 30) and a retinal prosthesis sensor-stimulator 1190 is positioned proximate the retina of a recipient. The external device 1110 and the processing module 1125 can communicate via coils 108, 20.

In an example, sensory inputs (e.g., photons entering the eye) are absorbed by a microelectronic array of the sensor-stimulator 1190 that is hybridized to a glass piece 1192 including, for example, an embedded array of microwires. The glass can have a curved surface that conforms to the inner radius of the retina. The sensor-stimulator 1190 can include a microelectronic imaging device that can be made of thin silicon containing integrated circuitry that convert the incident photons to an electronic charge.

The processing module 1125 includes an image processor 1123 that is in signal communication with the sensor-stimulator 1190 via, for example, a lead 1188 which extends through surgical incision 1189 formed in the eye wall. In other examples, processing module 1125 is in wireless communication with the sensor-stimulator 1190. The image processor 1123 processes the input into the sensor-stimulator 1190, and provides control signals back to the sensor-stimulator 1190 so the device can provide an output to the optic nerve. That said, in an alternate example, the processing is executed by a component proximate to, or integrated with, the sensor-stimulator 1190. The electric charge resulting from the conversion of the incident photons is converted to a proportional amount of electronic current which is input to a nearby retinal cell layer. The cells fire and a signal is sent to the optic nerve, thus inducing a sight perception.

The processing module 1125 can be implanted in the recipient and function by communicating with the external device 1110, such as a behind-the-ear unit, a pair of eyeglasses, etc. The external device 1110 can include an external light/image capture device (e.g., located in/on a behind-the-ear device or a pair of glasses, etc.), while, as noted above, in some examples, the sensor-stimulator 1190 captures light/images, which sensor-stimulator is implanted in the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

What is claimed is:

1. A method, comprising:
obtaining a plurality of clinical data sets, wherein each of the plurality of clinical data sets is associated with a respective one of a plurality of recipients of an implantable medical device;
determining a relative priority of clinical support of the plurality of recipients based, at least in part, on the plurality of clinical data sets; and
displaying, based on the relative priority of clinical support, a prioritized clinician task list.

2. The method of claim 1, wherein the implantable medical device comprises a hearing device and the plurality of clinical data sets comprise a plurality of fitting data sets.

3. The method of claim 1, wherein determining a relative priority of clinical support for the plurality of recipients comprises determining a relative priority of clinical support based on the plurality of clinical data sets and clinician data.

4. The method of claim 1, wherein the obtaining comprises receiving, via a wide area network, fitting data from one or more remote computing devices.

5. The method of claim 1, further comprising:
obtaining first clinical data applicable to a first time period and associated with a first recipient;
obtaining second clinical data applicable to a second time period and associated with the first recipient;
comparing the first clinical data to the second clinical data; and
determining changes in hearing performance based on the comparing, wherein the determining of the relative priority comprises determining a priority of the first recipient based on the changes in hearing performance.

6. The method of claim 1, wherein each of the plurality of clinical data sets indicates a physiological measurement of the respective recipient, and the determining of the relative priority is based on the physiological measurement.

7. The method of claim 1, wherein one or more of the plurality of clinical data sets indicates a request for assistance by a first recipient, wherein the determining of the relative priority is based on the request for assistance.

8. The method of claim 1, further comprising:
obtaining, in response to the displaying, operating parameters for the implantable medical device of a respective one of the plurality of recipients; and
providing the operating parameters to the implantable medical device of the respective one of the plurality of recipients to configure the operation of the implantable medical device of the respective one of the plurality of recipients.

9. The method of claim 1, further comprising:
receiving input from a user interface, the input defining an adjustment to an implantable medical device of a first recipient; and
communicating the adjustment to the implantable medical device of the first recipient.

10. The method of claim 9, wherein communicating the adjustment to the implantable medical device of the first recipient comprises providing an indication of the adjustment to a machine learning model, wherein the determination of the relative priority is based on the machine learning model.

11. The method of claim 10, further comprising modifying the machine learning model based on the indication.

12. The method of claim 1, further comprising:
obtaining preference information from a user, wherein the determining of the relative priority is further based on the preference information.

13-39. (canceled)

40. One or more non-transitory computer readable storage media comprising instructions that, when executed by a processor, cause the processor to:
obtain clinical data associated with a plurality of clinical profiles, wherein each of the plurality of clinical profiles is associated with a different medical device recipient;
analyze the clinical data to determine a relative priority between the plurality of clinical profiles; and
display, at a display screen, a prioritized task list in which the plurality of clinical profiles are organized based on the determined relative priority.

41. The one or more non-transitory computer readable storage media of claim 40, wherein the plurality of clinical profiles comprises a plurality of fitting profiles, the clinical data comprises fitting data and wherein the instructions that cause the processor to analyze the fitting data comprise instructions that cause the processor to:
analyze changes in hearing performance for one or more recipients.

42. The one or more non-transitory computer readable storage media of claim 40, wherein analyzing the clinical data comprises analyzing physiological measurements associated with one or more recipients.

43. The one or more non-transitory computer readable storage media of claim 40, further comprising instructions that cause the processor to:
obtain, in response to the displaying, operating parameters for an implantable medical device of a medical device recipient; and
provide the operating parameters to the implantable medical device of the medical device recipient to configure the operation of the implantable medical device of the medical device recipient.

44. The one or more non-transitory computer readable storage media of claim 40, wherein analyzing the clinical data comprises analyzing contextual data associated with one or more recipients.

45. The one or more non-transitory computer readable storage media of claim 40, further comprising instructions that cause the processor to:
receive input from a user interface, the input defining an adjustment to an implantable medical device of a first medical device recipient; and
communicate the adjustment to the first medical device of the first medical device recipient.

46. The one or more non-transitory computer readable storage media of claim 45, wherein instructions that cause the processor to communicate the adjustment to the implantable medical device of the first medical device recipient comprises instructions that cause the processor to:
provide an indication of the adjustment to a machine learning model, wherein the determined relative priority is based on the machine learning model.

47. The one or more non-transitory computer readable storage media of claim 46, further comprising instructions that cause the processor to:
modify the machine learning model based on the indication.
